SEQUENCE LISTING (38 sequences)

SEQ ID NO: 1 - DNA, 21 bp, Artificial Sequence
  Description of Artificial Sequence Synthetic primer
ctgagttagaattactctgaa

SEQ ID NO: 2 - DNA, 20 bp, Artificial Sequence
  Description of Artificial Sequence Synthetic primer
attcccatcgaccagagttg

SEQ ID NO: 3 - DNA, 20 bp, Artificial Sequence
  Description of Artificial Sequence Synthetic primer
tttgggatcacttcctcacc

SEQ ID NO: 4 - DNA, 20 bp, Artificial Sequence
  Description of Artificial Sequence Synthetic primer
gatcccatcgagaagaacca

SEQ ID NO: 5 - DNA, 20 bp, Artificial Sequence
  Description of Artificial Sequence Synthetic primer
acacctgcacgttctgtctg

SEQ ID NO: 6 - DNA, 20 bp, Artificial Sequence
  Description of Artificial Sequence Synthetic primer
atttggcacctatgcacctc

SEQ ID NO: 7 - DNA, 20 bp, Artificial Sequence
  Description of Artificial Sequence Synthetic primer
cacactgccatctgcaatct

SEQ ID NO: 8 - DNA, 20 bp, Artificial Sequence
  Description of Artificial Sequence Synthetic primer
gaagagcctgctttggacac

SEQ ID NO: 9 - DNA, 20 bp, Artificial Sequence
  Description of Artificial Sequence Synthetic primer
aaatttgagcgcagaaagga

SEQ ID NO: 10 - DNA, 20 bp, Artificial Sequence
  Description of Artificial Sequence Synthetic primer
gcaacgtggagtcggtttat

SEQ ID NO: 11 - DNA, 20 bp, Artificial Sequence
  Description of Artificial Sequence Synthetic primer
aggggcaaaaagttcttggt

SEQ ID NO: 12 - DNA, 20 bp, Artificial Sequence
  Description of Artificial Sequence Synthetic primer
gagttcgagaccagcctgac

SEQ ID NO: 13 - DNA, 20 bp, Artificial Sequence
  Description of Artificial Sequence Synthetic primer
gcttctcgggttcaagtgag

SEQ ID NO: 14 - DNA, 20 bp, Artificial Sequence
  Description of Artificial Sequence Synthetic primer
cccacctacgtactggccta

SEQ ID NO: 15 - DNA, 20 bp, Artificial Sequence
  Description of Artificial Sequence Synthetic primer
cttggccttgggtgtgtact

SEQ ID NO: 16 - DNA, 20 bp, Artificial Sequence
  Description of Artificial Sequence Synthetic primer
tggactcctctgtccagctt

SEQ ID NO: 17 - DNA, 20 bp, Artificial Sequence
  Description of Artificial Sequence Synthetic primer
ctagcgtgacccagaagagg

SEQ ID NO: 18 - DNA, 20 bp, Artificial Sequence
  Description of Artificial Sequence Synthetic primer
ggcaactcaagctcaaggac

SEQ ID NO: 19 - DNA, 20 bp, Artificial Sequence
  Description of Artificial Sequence Synthetic primer
actcagcgtggcttcttcat

SEQ ID NO: 20 - DNA, 20 bp, Artificial Sequence
  Description of Artificial Sequence Synthetic primer
ttttgctgctgttgttcctg

SEQ ID NO: 21 - DNA, 20 bp, Artificial Sequence
  Description of Artificial Sequence Synthetic primer
gatcgcactactgcactcca

SEQ ID NO: 22 - DNA, 20 bp, Artificial Sequence
  Description of Artificial Sequence Synthetic primer
cagcaatgcctttgtcttca

SEQ ID NO: 23 - DNA, 20 bp, Artificial Sequence
  Description of Artificial Sequence Synthetic primer
taccagccacaccagcatta

SEQ ID NO: 24 - DNA, 20 bp, Artificial Sequence
  Description of Artificial Sequence Synthetic primer
tcgcatgaaaacttgcgtag

SEQ ID NO: 25 - DNA, 20 bp, Artificial Sequence
  Description of Artificial Sequence Synthetic primer
ttgaagttccacagccactg

SEQ ID NO: 26 - DNA, 20 bp, Artificial Sequence
  Description of Artificial Sequence Synthetic primer
gccctctcctcatagtgctg

SEQ ID NO: 27 - DNA, 20 bp, Artificial Sequence
  Description of Artificial Sequence Synthetic primer
ccaggttcatgcctttttgt

SEQ ID NO: 28 - DNA, 19 bp, Artificial Sequence
  Description of Artificial Sequence Synthetic primer
ggctactcagctcgacctc

SEQ ID NO: 29 - DNA, 20 bp, Artificial Sequence
  Description of Artificial Sequence Synthetic primer
catcccactgtcaccagatg

SEQ ID NO: 30 - DNA, 20 bp, Artificial Sequence
  Description of Artificial Sequence Synthetic primer
taccagctcaccaagctcct

SEQ ID NO: 31 - DNA, 20 bp, Artificial Sequence
  Description of Artificial Sequence Synthetic primer
gcttcactgggtgtggaaat

SEQ ID NO: 32 - DNA, 20 bp, Artificial Sequence
  Description of Artificial Sequence Synthetic primer
ccaagttcatgctgtgtgct

SEQ ID NO: 33 - DNA, 20 bp, Artificial Sequence
  Description of Artificial Sequence Synthetic primer
agggagttgataggggtgct

SEQ ID NO: 34 - DNA, 19 bp, Artificial Sequence
  Description of Artificial Sequence Synthetic primer
tcaattcttgtccaatcca

SEQ ID NO: 35 - DNA, 20 bp, Artificial Sequence
  Description of Artificial Sequence Synthetic primer
ccatttttgcagttggaggt

SEQ ID NO: 36 - DNA, 20 bp, Artificial Sequence
  Description of Artificial Sequence Synthetic primer
ggagagggagcctgagaaac

SEQ ID NO: 37 - DNA, 19 bp, Artificial Sequence
  Description of Artificial Sequence Synthetic primer
tcgggagtgggtaatttgc

SEQ ID NO: 38 - protein, 4 aa, Artificial Sequence
  Description of Artificial Sequence Synthetic peptide
DEVD

(19) United States
(12) Patent Application Publication
(10) Pub. No.: US 2014/0348744 A1
(43) Pub. Date: Nov. 27, 2014

(54) COMPOSITIONS AND METHODS FOR REGULATING CANCER-RELATED SIGNALING PATHWAYS

(71) Applicant: Jacek K. Pinski, La Canada, CA (US)

(72) Inventor: Jacek K. Pinski, La Canada, CA (US)

(21) Appl. No.: 14/288,112

(22) Filed: May 27, 2014

Related U.S. Application Data
(60) Provisional application No. 61/827,451, filed on May 24, 2013.

(51) Int. Cl. C12N 15/113; C07K 16/28; G01N 33/50; C12Q 1/66; C12Q 1/68; G01N 33/574; A61K 47/48

(52) U.S. Cl. CPC C12N 15/1138; G01N 33/57434; C07K 16/2869; A61K 47/48638; C12Q 1/66; C12Q 1/6886; G01N 33/5011; C07K 2317/76; C12Q 2600/158; C12Q 2600/136
USPC 424/1.49; 435/375; 514/44 A; 435/7.23; 536/24.5; 424/174.1; 424/178.1; 435/6.13; 435/6.12; 506/9; 435/6.11

(57) ABSTRACT

The use of luteinizing hormone receptor (LHR) as a treatment target in cancer is provided. Data demonstrates that LHR plays an important role in androgen synthesis and signaling pathways critical for prostate cancer progression. When LHR is silenced, there is a significant downregulation of androgen receptor (AR) mRNA and protein expression with a subsequent suppression of PSA expression. Data suggesting that the LH-LHR signaling cascade may be an upstream and viable therapeutic target in castration-resistant prostate cancer (CRPC) is presented.

### CROSS-REFERENCE TO RELATED APPLICATION

This application claims priority under 35 U.S.C. §119(e) to U.S. Provisional Application No. 61/827,451, filed May 24, 2013, the content of which is hereby incorporated by reference into the present application.

### BACKGROUND

Aberrant androgen signaling is essential for the development and progression of prostate cancer. The backbone of systemic treatment for prostate cancer is androgen deprivation, targeting gonadal androgen synthesis. In the castration-resistant state, prostate cancer progresses despite the absence of gonadal androgens, but it is now apparent that androgen related gene networks remain active and androgen receptor (AR) expression persists throughout this progression. The androgens that initiate these cascades are synthesized by the adrenal glands and, importantly, by prostate cancer cells themselves. This is the rationale for novel agents targeting the androgen pathway, a strategy that has met with much success. Abiraterone, a CYP-17α inhibitor, was recently approved for use in castration-resistant prostate cancer (CRPC) and enzalutamide, an androgen receptor antagonist, has also garnered approval in this setting. While androgen synthesis and signaling are clearly viable therapeutic targets, the regulation of tumoral androgen synthesis remains largely undescribed.

Gonadal androgen synthesis is initiated when luteinizing hormone (LH) produced in the pituitary gland engages the LH receptor (LHR) on the cell surface of testicular Leydig cells. LHR is also expressed on the cell surface of hyperplastic and malignant prostate cells. While the presence of LHR on prostate cancer cells is well described, their function is unclear.

### SUMMARY OF THE DISCLOSURE

Provided herein are compositions and methods for achieving one or more of downregulating androgen receptor (AR) mRNA expression and/or AR protein expression; suppressing prostate (PSA) expression and/or PSA promoter activity; suppressing the activity of PKA; suppressing the activity of PI3K/AKT; suppressing the activity of ERK; or suppressing the expression of HER-2, in a cell expressing luteinizing hormone receptor (LHR), by contacting the cell with an effective amount of an agent that inhibits the expression of LHR. The contacting can be in vitro or in vivo. When the contacting is in vivo, the agent is administered locally or systemically. Non-limiting examples of agents useful in these methods include, for example, a small molecule inhibitor, an inhibitory RNA molecule or an anti-LHR antibody, antibody fragment or a derivative thereof. In one aspect the antibody is a monoclonal antibody or a fragment thereof.

When administered to a subject or patient, the invention also provide methods for inhibiting the growth of a cancer or tumor cell expressing LHR or alternatvely, for treating a cancer or tumor cell expressing LHR, in a subject in need of such treatment by administering to the subject an effective amount of an agent that inhibits the expression of LHR. The agent can be administered locally or systemically. Non-limiting examples of agents useful in these methods include, for example, a small molecule inhibitor, an inhibitory RNA molecule or an anti-LHR antibody or a derivative thereof. Non-limiting examples of tumor cells that express LHR include, for example, prostate cancer cell, castration-resistant prostate cancer cells (CRPC), breast cancer cells or ovarian cancer cells.

This disclosure also provides methods to screen for new potential therapeutic agents that possess one or more of downregulating androgen receptor (AR) mRNA expression and/or AR protein expression; suppressing prostate (PSA) expression and/or PSA promoter activity; suppressing the activity of PKA; suppressing the activity of PI3K/AKT; suppressing the activity of ERK; or suppressing the expression of HER-2. The method comprises, or alternatively consists essentially of, or yet further consists of, contacting a first cell expressing LHR with a test agent and noting the expression or activity of the one or more activities noted above and then comparing the noted expression or activity of one or more the noted activities with the expression or activity of that of a second cell expressing LHR having been contacted with an agent that inhibits the expression of LHR. If expression or activity of the first cell after contacting with the test agent is equal to or greater than that of the second cell, then the agent is a potential therapeutic agent.

Kits to perform the methods and/or screens are further provided herein.

The noted methods are based on the examined effect of LH exposure on prostate cancer cells wherein it was demonstrated that LH increases viability of LNCaP cells. Furthermore, these in vitro studies show that LH induces the expression of steroidogenic enzymes in LNCaP cells, resulting in the production of testosterone. This pathway is further characterized using small interfering RNA (siRNA) to silence LHR expression. In addition to its impact on cell viability and androgen synthesis, Applicant also reports the impact of LHR silencing on several key survival pathways in prostate cancer, including the MAPK and PKA signaling cascades.

### BRIEF DESCRIPTION OF THE DRAWINGS

FIGS. 1A to 1C show functional LHR expression in human prostate cancer tissue and LNCaP cell line, which was down-regulated by siRNA against human LHR. LHR expression was examined in tissues of 50 patients with prostate carcinoma by immunohistochemistry and in LNCAP cells by Western blot. LHR immunoreactive substance was demonstrated in human prostate carcinoma cells (arrowhead) (A), and in LNCAP prostate carcinoma cells (B), LNCAP cells were treated with human LH for 4 days, the phosphorylated PKA (p-PKA) was induced dose-dependently (1˜5 IU/ml) (C), confirming a functional role of LHR in these cells. LHR expression was significantly down-regulated after LNCaP cells were transfected for three days with siRNA against human LHR (B).

FIGS. 2A and 2B show silencing LHR prevents LH induced cell proliferation and increased apoptosis in LNCAP cells. LNCaP cells were transfected with siRNA against human LHR (LHR-siRNA) and compared with scramble siRNA as control, followed by LH treatment (1˜2 IU/ml) for five days. Cell proliferation was determined by MTS assay at 490 nm. Data was expressed as the mean±SD. Apoptosis was accessed by TUNEL staining and by colorimetric caspase 3 activity assay based on the hydrolysis of acetyl-Asp-Glu-Val-Asp p-nitroanilide (Ac-DEVD pNA) by caspase 3. The TUNEL labeled cells were counted in 10 randomly chosen fields (200×). Results were expressed as the percentage of TUNEL-positive cells/total number of cells counted. The result showed that silencing LHR prevents LH induced cell proliferation (p<0.01) (A) and increased apoptosis in LNCAP cells (B). LHR-siRNA transfected cells showed apoptotic morphology, i.e. cells displayed shrunken, became rounded, and stained dark detached (B). An induced caspase 3 activity was confirmed in LHR silenced LNCAP cells compared to control (p<0.01).

FIGS. 3A to 3C show silencing LHR suppresses androgen production in LNCaP cells. LHR-silenced LNCaP cells were analyzed for mRNA expression of androgen synthesis enzymes by Real time PCR and for androgen production by radioimmunoassay (RIA). A scramble siRNA sequence was used as the negative control. Data was expressed as the mean±SD from three experiments. Real time PCR assay was performed after three days' transfection of LNCAP cells with LHR-siRNA, revealing reduced expression of AKR1C1, AKR1C3 and CYP17A1 (p<0.05)(A) from the androgen enzymes tested. Androgen production was examined following two days' LHR-siRNA transfection and additional eight days' LH treatment (1.0 IU/ml) in RPMI 1640 medium containing 1% FBS. Testosterone and DHT were measure by RIA. Result showed that silencing LHR suppressed LH induced testosterone production in LNCaP cells (p<0.05) (B) and DHT became undetectable in LHR-silenced LNCAP cells (C).

FIGS. 4A and 4B show LHR silencing suppressed AR expression in LNCaP cells. LHR mRNA expression in LHR-silenced LNCAP cells was quantitatively measured by Real time PCR (A), and the protein expression, by Western blot (B). PCR amplifications for LHR and AR mRNA were performed on three days' LHR-siRNA transfection of the cells, showing both LHR (p<0.01) and AR (p<0.05) expressions were suppressed in the LHR-silenced LNCAP cells (A). The inhibitions of both LHR and AR transcripts were confirmed by the reduced LHR and AR protein expressions (B) accessed by Western blot assay on LHR-siRNA transfected LNCAP cells for four days.

FIGS. 5A to 5C show LHR silencing suppressed PSA expression and its promoter activity in LNCaP cells. PSA mRNA expression in LHR-silenced LNCAP cells was quantitatively measured by Real time PCR (A), and the protein expression, by Western blot (B). PCR amplification for PSA was performed on three days' LHR-siRNA transfection of the cells, showing decreased PSA expression in the LHR-silenced LNCAP cells (p<0.05) (A). The Western blot confirmed the reduced PSA protein expressions (B) in LHR-siRNA transfected LNCAP cells for four days. PSA promoter activity was examined with a dual-luciferase reporter assay by co-transfection of LNCaP cells with pGL3-PSA-luc/renilla-luc reporters. LHR silencing by three days transfection significantly suppressed LH-induced PSA promoter activity (LH 1-10 IU/ml, p<0.01). All values are standardized to Renilla luciferase activity (C).

FIGS. 6A to 6E show LHR silencing suppressed activity of PKA, PI3K/AKT, ERK and expression of HER-2. The activities of PKA, PI3K/AKT, ERK and expression HER-2 were accessed by Western blot on LHR-siRNa transfected LNCAP cells for four days. The data revealed a reduction of phosphorylated PKA catalytic unit (p-PKA) (A), phosphorylated ERK1/2/(p-ERK1/2) (B) and PI3K p85 subunit (PI3K/p85) (C). The activity of AKT2 was more dramatically decreased and not only phosphorylated AKT (p-AKT) but also total AKT2 was suppressed (D). The expression of HER-2 was also suppressed by LHR silencing (E).

FIG. 7 shows that LNCAP cells treated with control antibody supernatant and anti-LHR supernatant (5B1) for 4 days (1:1 volume ratio of cell culture media and antibody supernatants) were subjected to Western blot with anti-PI3 kinase P85 antibody (Cell signaling technology).

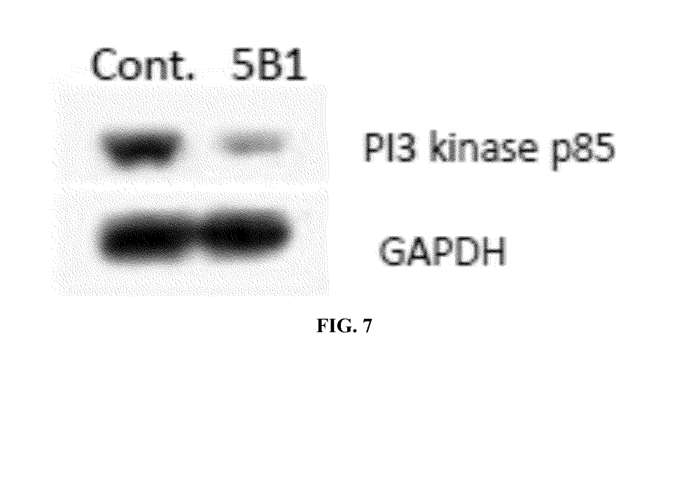

FIG. 8 shows the effects of anti-LHR supernatants on the growth of LNCaP cells. Cell proliferation of LNCAP cells treated with different anti-LHR supernatants for 4 days (1:1 volume ratio of cell culture media and anti-LHR supernatant) (* P value<0.01).

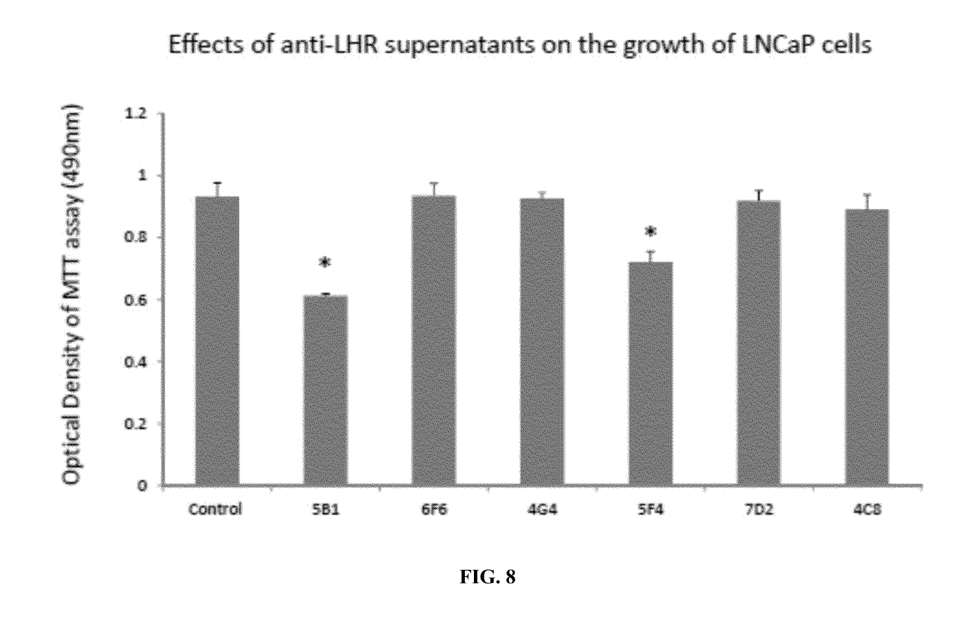

FIG. 9 shows the effects of anti-LHR supernatants on the proliferation of MCF-7 cells. Cell proliferation of MCF-7 cells treated with different anti-LHR supernatants for 4 days (1:1 volume ratio of cell culture media and anti-LHR supernatant) (* P value<0.01).

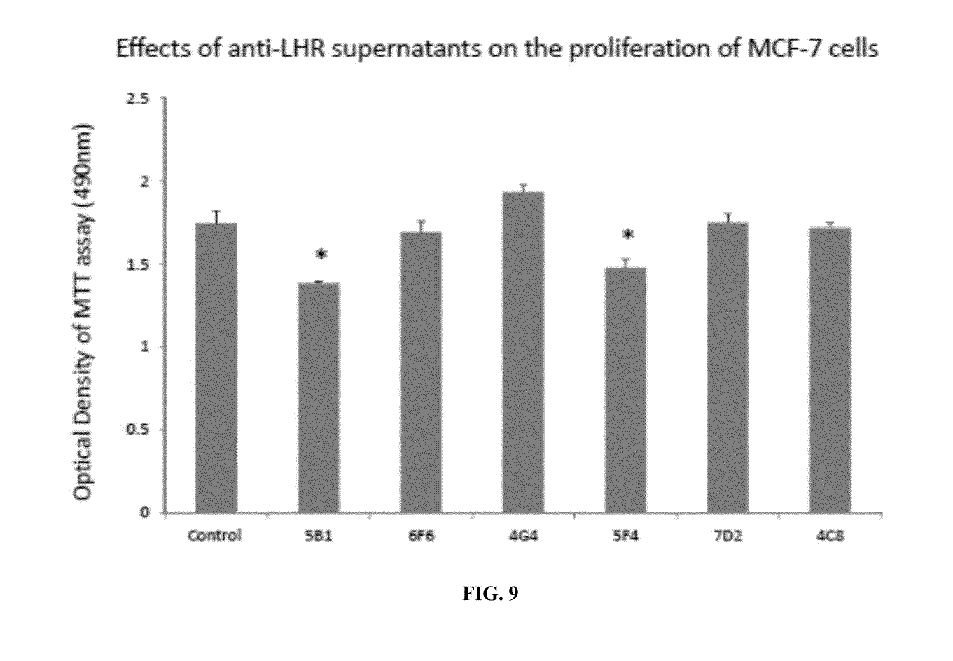

FIG. 10 shows the effects of anti-LHR supernatants on the growth of OV432 cells. Cell proliferation of OV432 cells treated with different anti-LHR supernatants for 4 days (1:1 volume ratio of cell culture media and anti-LHR supernatants) (* P value<0.01).

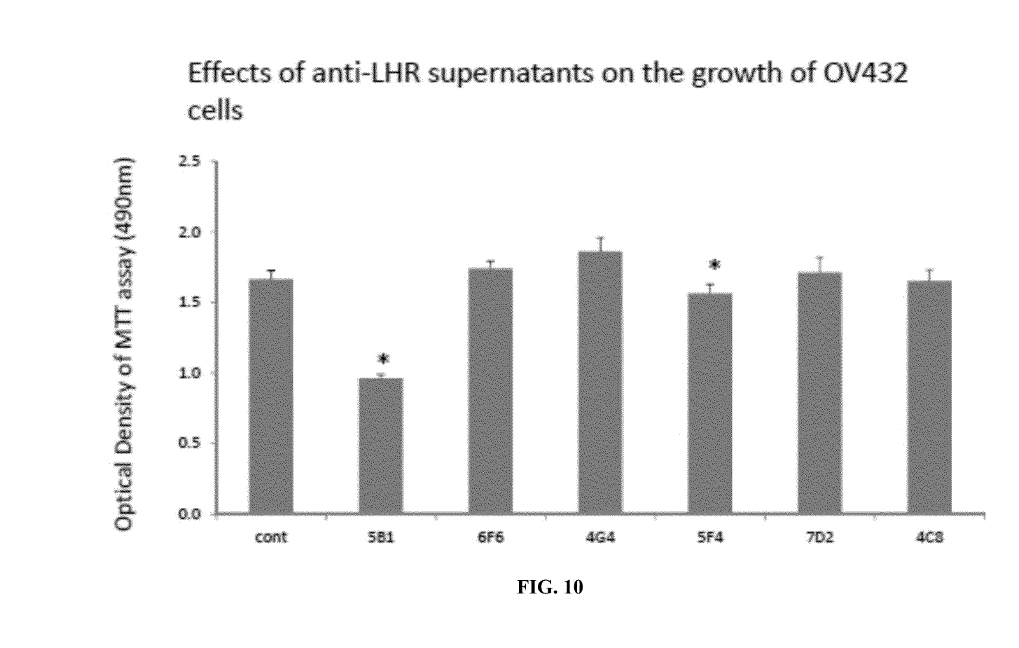

### DETAILED DESCRIPTION

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; and Mayer and Walker eds. (1987) Immunochemical Methods in Cell and Molecular Biology (Academic Press, London).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

### DEFINITIONS

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “binding” or “binds” as used herein are meant to include interactions between molecules that may be detected using, for example, a hybridization assay or a Western blot. The terms are also meant to include “binding” interactions between molecules. Interactions may be, for example, protein-protein, antibody-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. The term “peptide fragment,” as used herein, also refers to a peptide chain.

The phrase “equivalent polypeptide” or “equivalent peptide fragment” refers to protein, polynucleotide, or peptide fragment which hybridizes to the exemplified polynucleotide or peptide fragment under stringent conditions and which exhibit similar biological activity in vivo, e.g., approximately 100%, or alternatively, over 90% or alternatively over 85% or alternatively over 70%, as compared to the standard or control biological activity. Additional embodiments within the scope of this invention are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Biologically equivalent polynucleotides are those having the specified percent homology and encoding a polypeptide having the same or similar biological activity.

The term “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “probe” when used in the context of polynucleotide manipulation refers to an oligonucleotide that is provided as a reagent to detect a target potentially present in a sample of interest by hybridizing with the target. Usually, a probe will comprise a detectable label or a means by which a label can be attached, either before or subsequent to the hybridization reaction. Alternatively, a “probe” can be a biological compound such as a polypeptide, antibody, or fragments thereof that is capable of binding to the target potentially present in a sample of interest.

“Detectable labels” or “markers” include, but are not limited to radioisotopes, alpha emitters, fluorochromes, chemiluminescent compounds, dyes, and proteins, including enzymes. Detectable labels can also be attached to a polynucleotide, polypeptide, antibody or composition described herein.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). All processes of producing replicate copies of a polynucleotide, such as PCR or gene cloning, are collectively referred to herein as “replication.” A primer can also be used as a probe in hybridization reactions, such as Southern or Northern blot analyses. Sambrook and Russell (2001), infra.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Additional examples of stringent hybridization conditions include: low stringency of incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “propagate” or “expand” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this invention are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the invention may be based on a genetically or otherwise (e.g. by specific choice of packaging cell system) altered version of a particular retrovirus.

That the vector particle according to the invention is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “promoter” refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. An inducible promoter is one which is capable of being induced by a molecule or a factor added to the cell or expressed in the cell. An inducible promoter may still produce a basal level of transcription in the absence of induction, but induction typically leads to significantly more production of the protein. Promoters can also be tissue specific. A tissue specific promoter allows for the production of a protein in a certain population of cells that have the appropriate transcriptional factors to activate the promoter.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

Antibodies of the present invention include naturally purified products, products of chemical synthetic procedures, and products produced by recombinant techniques from a eukaryotic host, including, for example, yeast, higher plant, insect and mammalian cells, or alternatively from a prokaryotic cells as described above.

As used herein and unless specifically noted, an “antibody” includes whole antibodies, any antigen binding fragment, antibody derivative or variant thereof, as well as human or haumanized antibodies or a single chain thereof. Unless specifically stated, the term “antibody” includes polyclonal antibodies, monoclonal antibodies as well as any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention. The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the V_{L}, V_{H}, C_{L} and CH, domains; a F(ab′)2 fragment, a bivalent fragment comprising two Fab fragments linked by a disulfide bridge at the hinge region; a Fd fragment consisting of the V_{H} and C_{H}, domains; a Fv fragment consisting of the V_{L} and V_{H} domains of a single arm of an antibody, a dAb fragment (Ward et al. (1989) Nature 341:544-546), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). Bird et al. (1988) Science 242:423-426 and Huston et al. (1988) Proc. Natl. Acad. Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

Examples of binding fragments encompassed within the term “antigen binding portion” of an antibody include a Fab fragment, a monovalent fragment consisting of the V_{L}, V_{H}, C_{L} and CH, domains; a F(ab′)2 fragment, a bivalent fragment comprising two Fab fragments linked by a disulfide bridge at the hinge region; a Fd fragment consisting of the V_{H} and C_{H}, domains; a Fv fragment consisting of the V_{L} and V_{H} domains of a single arm of an antibody, a dAb fragment (Ward et al. (1989) Nature 341:544-546), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). Bird et al. (1988) Science 242:423-426 and Huston et al. (1988) Proc. Natl. Acad. Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “antibody” also includes antibody derivatives. The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, trispecific, tetraspecific, multispecific antibodies, diabodies, chimeric, recombinant and humanized. The term also includes antibodies produced in a species other than a mouse. It also includes antibodies containing post-translational modifications to the linear polypeptide sequence of the antibody or fragment. It further encompasses fully human antibodies.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

The term “antibody” also includes bispecific molecules”. The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “antibody” also includes heteroantibodies. The term “heteroantibodies” refers to two or more antibodies, antibody binding fragments (e.g., Fab), derivatives thereof, or antigen binding regions linked together, at least two of which have different specificities.

The term “antibody” also includes a human antibody. The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

An antibody also includes antibodies derived from a particular germline sequence. As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

An antibody also includes a human monoclonal antibody. A “human monoclonal antibody” refers to antibodies displaying a single binding specificity which have variable and constant regions derived from human germline immunoglobulin sequences.

An antibody also includes a recombinant human antibody. The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A population of cells, polynucleotides (e.g., DNA or RNA), antibodies, polypeptides or proteins intends a collection of more than one composition. When referring to a population of cells, the population can be completely identical (clonal) or non-identical in phenotype and/or genotype. A substantially homogenous population of a composition (e.g., cells polynucleotides (e.g., DNA or RNA), antibodies, polypeptides or proteins) is a population having at least 70%, or alternatively at least 75%, or alternatively at least 80%, or alternatively at least 85%, or alternatively at least 90%, or alternatively at least 95%, or alternatively at least 98% identity.

As used herein the term “labile linker” intends a small molecule, amino acid, peptides that form reversible covalent bonds, pH sensitive linkages (acid or base sensitive), enzyme sensitive linkages, degradation sensitive linkers, photosensitive linkers, and the like, and combinations thereof that attach two components to each other for ease of delivery but under appropriate conditions, release the components from each other. For example, a labile linker cab be designed to direct release in a particular intracellular compartment or in an extracellular compartment in which antibody targeting compounds may accumulate. An acid-labile linker such as a cis-aconitic acid linker can take advantage of the acidic environment of different intracellular compartments such as the endosomes encountered during receptor mediated endocytosis and the lysosomes. EP 0495255B1 and Ducry and Stump (2010) Bioconjugate Chem. 21:5-13, disclose examples of several labile and non-labile linkers. As is apparent to the skilled artisan, a non-labile linker is a small molecule, amino acid or peptides that do not form reversible covalent bonds, pH sensitive linkages (acid or base sensitive), enzyme sensitive linkages, degradation sensitive linkers, photosensitive linkers, and the like, and combinations thereof that attach two components to each other for ease of delivery but under appropriate conditions but do not release the components from each other.

A “composition” is intended to mean a combination of active polypeptide, polynucleotide or antibody and another compound or composition, inert (e.g. a detectable label) or active (e.g. a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like. In one embodiment, the mammals include horses, dogs, and cats.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks.

As used herein, the term “disease or condition characterized by expression on a cancer or tumor cell” intends prostate cancers, breast cancers, including triple negative breast cancer, bladder, colorectal and pancreatic cancers, sarcomas, lymphomas, melanomas and renal cell carcinomas.

The term administration shall include without limitation, local and systemic administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

### MODES FOR CARRYING OUT THE DISCLOSURE

This disclosure provides compositions and methods for achieving one or more of downregulating androgen receptor (AR) mRNA expression and/or AR protein expression; suppressing prostate (PSA) expression and/or PSA promoter activity; suppressing the activity of PKA; suppressing the activity of PI3K/AKT; suppressing the activity of ERK (ERK1/2); or suppressing the expression of HER-2, in a cell expressing luteinizing hormone receptor (LHR), by contacting the cell with an effective amount of an agent that inhibits the expression of LHR. The contacting can be in vitro or in vivo.

When the contacting is in vitro, the method provides a screen for identifying potential therapeutic agents for use in the therapeutic methods as disclosed herein. For example, to screen for new potential therapeutic agents that possess one or more of downregulating androgen receptor (AR) mRNA expression and/or AR protein expression; suppressing prostate (PSA) expression and/or PSA promoter activity; suppressing the activity of PKA; suppressing the activity of PI3K/AKT; suppressing the activity of ERK; or suppressing the expression of HER-2, a first cell expressing LHR is contacted with a potential therapeutic agent and then noting and comparing the expression or activity of one or more of the noted activities with the reported and noted expression or activity of that of a second cell expressing LHR that was with an agent that is known to inhibit the expression of LHR (e.g., an anti-LHR inhibitory RNA or anti-LHR antibody), wherein if the reported expression or activity of the first cell is equal to or greater than that of the second cell, the agent is a potential therapeutic agent. The potential therapeutic agent can then be tested using a pre-clinical animal model known in the art or as described herein.

To perform the screen, the cells expressing LHR are cultured in the presence of the agents and the effect on one or more of: LHR expression, AR mRNA expression, AR protein expression, PSA expression, PSA promoter activity, activity of PKA, the activity of PI3K/AKT, the activity of ERK1/2, the expression of HER-2, is noted using one or more method known in the art, e.g., by PCR, real-time PCR, Western blot, immunohistochemical analysis, TUNEL, luciferase assay for PSA promoter activity, or cell growth, death or apoptosis. Where appropriate, the agents can be added to the cell culture medium and introduced into the cells using conventional gene transfer techniques and vectors. The agents can be added directly or combined with a carrier, such as a pharmaceutically acceptable carrier for administration.

Suitable cells for use in the screening method include cells of any appropriate species, e.g., mammalian such as insect, murine, bovine, canine, equine, feline and human cells. The cells can be isolated from a human tumor biopsy and therefore can provide personalized therapy for the individual to determine if a candidate therapeutic will be appropriate for the patient. Alternatively, the cells can be cultured cell lines (e.g., PC LNCaP cells purchased from the American Type Culture Collection (ATCC), ovarian cancer cells or breast cancer cells expressing LHR and commercially available, e.g., from the ATCC) and maintained in the appropriate culture medium.

When the contacting is in vivo, the methods are useful therapeutically or to screen for potential therapeutics by administration to an appropriate animal model. Establishment of human prostate cancer xenograft is described below as an example of an appropriate animal model. While the implanted cells are prostate cancer cells, other cancer cells, e.g., breast or ovarian cancer cells, can be similarly utilized.

In the animal model, the agent is administered locally or systemically using methods known in the art and briefly described herein. An agent is considered to be a potential therapeutic by a reduction in the tumor mass or by sampling the tumor for reduced expression of AR and/or LHR protein or mRNA. Immunohistochemical or molecular techniques known in the art can be applied for this analysis and are briefly described herein.

When administered to a subject or patient, the methods also provide methods for treating a cancer or tumor expressing LHR in a subject in need of such treatment by administering to the subject an effective amount of an agent that inhibits the expression of LHR. As reported in Engel et al. (2012) Expert. Opin. Investig. Drugs, 21(6):891-899, LHR is expressed in 86% of prostate cancers, and about 50% of breast cancers, including triple negative breast cancer, as well as bladder, colorectal and pancreatic cancers, sarcomas, lymphomas, melanomas and renal cell carcinomas. Thus, the compositions and methods as disclosed herein are useful in treating a patient or subject suffering from a cancer characterized by cells expressing LHR.

Prior to treating the patient, the method can optionally comprise testing a tumor sample isolated from the patient and determining by a method known in the art (e.g., immunohistochemistry) to determine if the tumor expresses LHR. Tumors expressing LHR are preferentially treated by the therapeutic methods disclosed herein.

The agent can be administered locally or systemically. Non-limiting examples of agents useful in these methods include, for example, a small molecule inhibitor (e.g., AEZS-108, as described in Engel et al. (2012) supra.), an inhibitory RNA molecule or an anti-LHR antibody or a derivative thereof.

In one aspect in connection with the above methods when the agent is an antibody, antibody fragment or antibody derivative, the anti-LHR antibody can be combined with a radiolabel, e.g., 1-131, Y-90, or an alpha emitter such as such Bi-213 or Th227. Alternatively, the antibody, fragment or derivative is a fusion polypeptide combined with a cytokine (e.g., IL-2, IL-15, IL21, IL-12, IFNg, IFNalpha) or chemokines (e.g., LEC) or co-stimulatory molecules (e.g., CD137L, B7.1, GITRL, CD40L or OX40L). Methods for manufacturing such are known in the art and described for example in U.S. Published Application Nos. 2011/0002921 and 2014/0099305). In addition, the anti-LHR antibody can be conjugated to a second or third therapeutic agent (e.g., a small molecule) with cleavable or non-cleavable linkers or alternatively, labile or non-labile linkers.

Non-limiting examples of linkers for making antibody-drug conjugates (ADCs) in this invention include cleavable and non-cleavable linkers. For example, brentuximab vedotin (ADCETRIS®; Seattle Genetics) features the cleavable linker vcMMAE, while ado-trastuzumab emtansine (Kadcyla™; Genentech/Roche) contains the non-cleavable linker SMCC. In either case, the stability of the ADC during delivery to the target site is key to achieving a desirable therapeutic index. Cleavable linkers take advantage of the ADC targeting mechanism which involves sequential binding of the ADC to its cognate antigen on the surface of the target cancer cells, and internalization of the ADC-antigen complexes through the endosomal-lysosomal pathway. Intracellular liberation of the cytotoxin in these cases relies on the fact that endosomes/lysosomes are acidic compartments that will facilitate cleavage of acid-labile chemical linkages such as hydrazone. In addition, if a lysosomal-specific protease cleavage site is engineered into the linker, for example the cathepsin B site in vcMMAE, the cytotoxins will be liberated in proximity to their intracellular targets. Alternatively, linkers containing mixed disulfides provide yet another approach by which cytotoxic payloads can be liberated intracellularly as they are selectively cleaved in the reducing environment of the cell, but not in the oxygen-rich environment in the bloodstream. In contrast, non-cleavable linkers liberate toxic payloads during lysosomal degradation of the ADC within the target cell.

Non-limiting examples of such diseases or conditions include, for example, prostate cancer, castration-resistant prostate cancer (CRPC), breast cancers, including triple negative breast cancer, as well as ovarian, bladder, colorectal and pancreatic cancers, sarcomas, lymphomas, melanomas and renal cell carcinomas.

Kits to perform the methods and/or screens are further provided herein. The kits comprise are useful in one or more of androgen receptor (AR) mRNA expression and/or AR protein expression; prostate (PSA) expression and/or PSA promoter activity; the activity of PKA; the activity of PI3K/AKT; the activity of ERK; or the expression of HER-2, comprising, or alternatively consisting essentially of, or yet further consisting of, an effective amount of an agent that inhibits the expression of LHR and optionally instructions for use. Non-limiting examples of the agent that inhibits the expression of LHR is a small molecule inhibitor, an inhibitory RNA molecule, or an anti-LHR antibody or a fragment or derivative thereof.

### Inhibitory RNA

In one aspect, the agent that inhibits the expression of LHR is an inhibitory RNA molecule, e.g., e.g. antisense RNA or siRNA. Non-limiting examples of such are inhibitory RNA complementary to SEQ ID NO: 1 (CTGAGTTAGAATTACTCTGAA). In another aspect, the inhibitory RNA or their biological equivalents are labeled with a detectable marker or label, such as a dye or radioisotope, for ease of detection.

This disclosure also provides isolated DNA sequences that encode the inhibitory RNA sequence. The polynucleotides (DNA and RNA) of this invention can be replicated using conventional recombinant techniques. Alternatively, DNA polynucleotides can be replicated using PCR technology. PCR is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) and references cited therein. In a separate embodiment, these polynucleotides are further isolated. Still further, one of skill in the art can operatively link the polynucleotides to regulatory sequences (e.g., promoters, enhancers, or expression or delivery vehicles or vectors) for their expression in a host cell for replication or expression of the inhibitory RNA sequences. The polynucleotides and regulatory sequences can be are inserted into the host cell (prokaryotic or eukaryotic) for replication and amplification. The polynucleotides can be isolated from the cell by methods well known to those of skill in the art. A process for obtaining polynucleotides by this method is further provided herein as well as the polynucleotides so obtained.

In one aspect, the RNA is short interfering RNA, also known as siRNA. Methods to prepare and screen interfering RNA and select for the ability to block polynucleotide expression are known in the art and non-limiting examples of which are shown below. These interfering RNA are provided by this invention alone or in combination with a suitable vector for insertion and expression in a host cell. Compositions containing the siRNA and DNA encoding such are further provided.

siRNA sequences can be designed by obtaining the target LHR mRNA sequence and determining an appropriate siRNA complementary sequence. siRNAs of the invention are designed to interact with a target sequence, e.g., LHR, meaning they complement a target sequence sufficiently to hybridize to that sequence. An siRNA can be 100% identical or complimentary to the target sequence. However, homology of the siRNA sequence to the target sequence can be less than 100% as long as the siRNA can hybridize to the target sequence. Thus, for example, the siRNA molecule can be at least 70%, 75%, 80%, 85%, 90%, 95%, 96%, 97%, 98%, 99% or 100% identical to the target sequence or the complement of the target sequence. Therefore, siRNA molecules with insertions, deletions or single point mutations relative to a target may also be used. The generation of several different siRNA sequences per target mRNA is recommended to allow screening for the optimal target sequence. A homology search, such as a BLAST search, should be performed to ensure that the siRNA sequence does not contain homology to any known mammalian gene.

Researchers have determined that certain characteristics are common in siRNA molecules that effectively silence their target gene (Duxbury (2004) J. Surgical Res. 117:339-344; Ui-Tei et al. (2004) Nucl. Acids Res. 32:936-48). As a general guide, siRNAs that include one or more of the following conditions are particularly useful in gene silencing in mammalian cells: GC ratio of between 45-55%, no runs of more than 9 G/C residues, G/C at the 5′ end of the sense strand; A/U at the 5′ end of the antisense strand; and at least 5 A/U residues in the first 7 bases of the 5′ terminal of the antisense strand.

siRNA are, in general, from about 10 to about 30 nucleotides in length. For example, the siRNA can be 10-30 nucleotides long, 12-28 nucleotides long, 15-25 nucleotides long, 19-23 nucleotides long, or 21-23 nucleotides long. When an siRNA contains two strands of different lengths, the longer of the strands designates the length of the siRNA. In this situation, the unpaired nucleotides of the longer strand would form an overhang.

The term siRNA includes short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides long. For example, the stem can be 10-30 nucleotides long, 12-28 nucleotides long, 15-25 nucleotides long, 19-23 nucleotides long, or 21-23 nucleotides long.

Tools to assist siRNA design are readily available to the public. For example, a computer-based siRNA design tool is available on the internet at www.dharmacon.com, last accessed on Nov. 26, 2007.

This invention also provides compositions for in vitro and in vivo use comprising, or alternatively consisting essentially of, or yet further consisting of one or more of the isolated polynucleotide as described herein and a pharmaceutically acceptable carrier. In one aspect, the compositions are pharmaceutical formulations for use in the therapeutic methods of this invention.

### Synthesis of siRNA

siRNA can be synthesized chemically or enzymatically in vitro as described in Micura (2002) Agnes Chem. Int. Ed. Emgl. 41:2265-2269; Betz (2003) Promega Notes 85:15-18; and Paddison and Hannon (2002) Cancer Cell. 2:17-23. Chemical synthesis can be performed via manual or automated methods, both of which are well known in the art as described in Micura (2002), supra. siRNA can also be endogenously expressed inside the cells in the form of shRNAs as described in Yu et al. (2002) Proc. Natl. Acad. Sci. USA 99:6047-6052; and McManus et al. (2002) RNA 8:842-850. Endogenous expression has been achieved using plasmid-based expression systems using small nuclear RNA promoters, such as RNA polymerase III U6 or H1, or RNA polymerase II U1 as described in Brummelkamp et al. (2002) Science 296:550-553 (2002); and Novarino et al. (2004) J. Neurosci. 24:5322-5330.

In vitro enzymatic siRNA synthesis can be performed using an RNA polymerase mediated process to produce individual sense and antisense strands that are annealed in vitro prior to delivery into the cells of choice as describe in Fire et al. (1998) Nature 391:806-811; Donze and Picard (2002) Nucl. Acids Res. 30(10):e46; Yu et al. (2002); and Shim et al. (2002) J. Biol. Chem. 277:30413-30416. Several manufacturers (Promega, Ambion, New England Biolabs, and Stragene) produce transcription kits useful in performing the in vitro synthesis.

In vitro synthesis of siRNA can be achieved, for example, by using a pair of short, duplex oligonucleotides that contain T7 RNA polymerase promoters upstream of the sense and antisense RNA sequences as the DNA template. Each oligonucleotide of the duplex is a separate template for the synthesis of one strand of the siRNA. The separate short RNA strands that are synthesized are then annealed to form siRNA as described in Protocols and Applications, Chapter 2: RNA interference, Promega Corporation, (2005).

RNA also can be obtained by first inserting a DNA polynucleotide encoding the RNA into a suitable prokaryotic or eukaryotic host cell. The DNA can be operatively linked to appropriate regulatory sequences and the heterologous DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook and Russell (2001) supra. For instance, mRNA can be isolated using various lytic enzymes or chemical solutions according to the procedures set forth in Sambrook and Russell (2001) supra or extracted by nucleic-acid-binding resins following the accompanying instructions provided by manufactures.

The present invention also provides delivery vehicles suitable for delivery of a polynucleotide of the invention into cells (whether in vivo, ex vivo, or in vitro). A polynucleotide of the invention can be contained within a gene delivery vehicle, a cloning vector or an expression vector. These vectors (especially expression vectors) can in turn be manipulated to assume any of a number of forms which may, for example, facilitate delivery to and/or entry into a cell.

As noted above, the invention further provides the isolated polynucleotides of this invention operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see Gene Expression Technology (Goeddel ed., Academic Press, Inc. (1991)) and references cited therein and Vectors: Essential Data Series (Gacesa and Ramji, eds., John Wiley & Sons, N.Y. (1994)), which contains maps, functional properties, commercial suppliers and a reference to GenEMBL accession numbers for various suitable vectors. Preferable, these vectors are capable of transcribing RNA in vitro or in vivo.

Expression vectors containing these nucleic acids are useful to obtain host vector systems to produce polynucleotides, proteins and polypeptides, for example, the antibodies, fragments or derivative thereof as described above. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the polypeptide or protein can be recombinantly produced. Suitable host cells will depend on the vector and can include mammalian cells, animal cells, human cells, simian cells, insect cells, yeast cells, and bacterial cells as described above and constructed using well known methods. See Sambrook and Russell (2001), supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; DEAE-dextran; electroporation; or microinjection. See Sambrook and Russell (2001), supra for this methodology.

Operatively linked to polynucleotides are sequences necessary for the translation and proper processing of the peptides. Examples of such include, but are not limited to a eukaryotic promoter, an enhancer, a termination sequence and a polyadenylation sequence. Construction and use of such sequences are known in the art and are combined with IRES elements and protein sequences using recombinant methods. “Operatively linked” shall mean the juxtaposition of two or more components in a manner that allows them to junction for their intended purpose. Promoters are sequences which drive transcription of the marker or target protein. It must be selected for use in the particular host cell, i.e., mammalian, insect or plant. Viral or mammalian promoters will function in mammalian cells. The promoters can be constitutive or inducible, examples of which are known and described in the art.

These isolated host cells containing the polynucleotides of this invention are useful in the methods described herein as well as for the recombinant replication of the polynucleotides and for the recombinant production of peptides and for high throughput screening.

### Host Cells

Also provided are host cells comprising one or more of the polynucleotides or antibodies of this invention. Suitable cells include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escerichia coli, Salmonella enterica and Streptococcus gordonii. The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

In addition to species specificity, the cells can be of any particular tissue type such as animal, mammalian, e.g., simian, bovine, canine, equine, feline, rat, muring or human.

### Therapeutic Antibody Compositions

This invention also provides an antibody, fragment or derivative of each thereof, capable of specifically forming a complex with LHR, which are useful in the methods of this disclosure. The term “antibody” includes polyclonal antibodies and monoclonal antibodies, antibody fragments, as well as derivatives thereof (described above). The antibodies include, but are not limited to mouse, rat, and rabbit or human antibodies. Also provided are hybridoma cell lines producing monoclonal antibodies of this invention.

Polyclonal antibodies of the invention can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, which induces the B-lymphocytes to produce IgG immunoglobulins specific for the antigen. This IgG is purified from the mammal's serum. Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a slow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods described in U.S. Pat. Nos. 7,279,559; 7,119,179; 7,060,800; 6,709,659; 6,656,746; 6,322,788; 5,686,073; and 5,670,153.

The monoclonal antibodies of the invention can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived there from, or any other suitable cell line as known in the art (see, e.g., www.atcc.org, www.lifetech.com., last accessed on Nov. 26, 2007, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) Bioinvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1977) Microbiol. Immunol. 41:901-907 (1997); Sandhu et al. (1996) Crit. Rev. Biotechnol. 16:95-118; Eren et al. (1998) Immunol. 93:154-161 that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al., Proc. Natl. Acad. Sci. USA (1996) 93:7843-7848); gel microdroplet and flow cytometry (Powell et al. (1990) Biotechnol. 8:333-337; One Cell Systems, (Cambridge, Mass.); Gray et al. (1995) J. 1 mm. Meth. 182:155-163; and Kenny et al. (1995) Bio. Technol. 13:787-790); B-cell selection (Steenbakkers et al. (1994) Molec. Biol. Reports 19:125-134.

Antibodies, fragments or derivatives thereof can also be prepared by delivering a polynucleotide encoding an antibody, fragment or derivative thereof of this invention to a suitable host cell for recombinant production as described above and known in the art or alternatively by delivering such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody derivatives also can be prepared by delivering a polynucleotide encoding the derivative to provide transgenic plants and cultured plant cells (e.g., but not limited to tobacco, maize, and duckweed) that produce such antibodies, specified portions or variants in the plant parts or in cells cultured there from. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies of the present invention can also be produced using transgenic plants, according to known methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present invention can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See, e.g., Russel et al. (2000) Infection and Immunity April 2000:1820-1826; Gallo et al. (2000) European J. of Immun. 30:534-540; Green (1999) J. of Immun. Methods 231:11-23; Yang et al. (1999) J. of Leukocyte Biology 66:401-410; Yang (1999) Cancer Research 59(6):1236-1243; Jakobovits (1998) Advanced Drug Delivery Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies of this invention also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies of this invention can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Hollinger et al., (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.)

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Fd segments (V_{H}-C_{H}1-VH-C_{H}1) which form a pair of antigen binding regions. Linear antibodies can be bispecific or monospecific.

The antibodies of this invention can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

If a monoclonal antibody being tested binds with protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski, et al. (1985) Proc. Natl. Acad. Sci. USA 82:8653 or Spira, et al. (1984) J. Immunol. Methods 74:307.

The isolation of other hybridomas secreting monoclonal antibodies with the specificity of the monoclonal antibodies of the invention can also be accomplished by one of ordinary skill in the art by producing anti-idiotypic antibodies. Herlyn, et al. (1986) Science 232:100. An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

Antibodies can be conjugated, for example, to a pharmaceutical agent, such as chemotherapeutic drug or a toxin or a radioisotope, cytokine, chemokine, co-stimulatory molecule and linked by cleavable, non-cleavable, labile or non-labile linkers.

The antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

### Compositions for Therapy

One or more of the above small molecule, antibody, antibody fragment, antibody derivative, siRNA, host cell, polynucleotide encoding these compositions can be further combined with each other and/or a carrier, a pharmaceutically acceptable carrier or medical device which is suitable for use of the compositions in screening or therapeutic methods. Thus, the compositions comprise, or alternatively consist essentially of, or yet further consists of, one or more of the above compositions described above in combination with each other and alternatively, a carrier such as a pharmaceutically acceptable carrier or medical device.

The carrier can be a liquid phase carrier or a solid phase carrier, e.g., bead, gel, microarray, or carrier molecule such as a liposome. The composition can optionally further comprise at least one further compound, protein or composition.

Additional examples of “carriers” includes therapeutically active agents such as another peptide or protein (e.g., an Fab′ fragment). For example, an antibody of this invention, derivative or fragment thereof can be functionally linked (e.g., by chemical coupling, genetic fusion, noncovalent association or otherwise) to one or more other molecular entities, such as another antibody (e.g., to produce a bispecific or a multispecific antibody), a cytotoxin, a cellular ligand or an antigen. Accordingly, this invention encompasses a large variety of antibody conjugates, bi- and multispecific molecules, and fusion proteins, whether or not they target the same epitope as the antibodies of this invention.

Yet additional examples of carriers are organic molecules (also termed modifying agents) or activating agents, that can be covalently attached, directly or indirectly, to an antibody of this invention. Attachment of the molecule can improve pharmacokinetic properties (e.g., increased in vivo serum half-life). Examples of organic molecules include, but are not limited to a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane.

Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. A suitable hydrophilic polymer that modifies the antibody of the invention has a molecular weight of about 800 to about 150,000 Daltons as a separate molecular entity. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Examples of such include, but are not limited to n-dodecanoate, n-tetradecanoate, n-octadecanoate, n-eicosanoate, n-docosanoate, n-triacontanoate, n-tetracontanoate, cis-Δ9-octadecanoate, all cis-Δ5,8,11,14-eicosatetraenoate, octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The present invention provides a composition comprising, or alternatively consisting essentially of, or yet further consisting of, at least one antibody of this invention, derivative or fragment thereof, suitable for administration in an effective amount to increase or induce cell cancer death, eliminate viral particles associated with a viral infection, and/or treat or ameliorate a neurodegenerative disease. The compositions include, for example, pharmaceutical and diagnostic compositions/kits, comprising a pharmaceutically acceptable carrier and at least one antibody of this invention, variant, derivative or fragment thereof. As noted above, the composition can further comprise additional antibodies or therapeutic agents which in combination, provide multiple therapies tailored to provide the maximum therapeutic benefit.

Alternatively, a composition of this invention can be co-administered with other therapeutic agents, whether or not linked to them or administered in the same dosing. They can be co-administered simultaneously with such agents (e.g., in a single composition or separately) or can be administered before or after administration of such agents.

The disclosure also provides an article of manufacture, comprising packaging material and at least one vial comprising a solution of at least one agent or composition with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising at least one agent or composition and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

Formulations of the present invention can be prepared by a process which comprises mixing at least one agent or composition and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of agent or composition that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include pen-injector devices for delivery of a solution such as BD Pens, BD Autojectore, Humaject®, NovoPen®, B-D® Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Methods of delivery include but are not limited to local or systemic, e.g., intra-arterial, intra-muscular, and intravenous. In a specific embodiment, it may be desirable to administer the pharmaceutical compositions of the disclosure locally to the area in need of treatment; this may be achieved by, for example, and not by way of limitation, local infusion during surgery, by injection or by means of a catheter. In some embodiments, the compositions are administered by intravenous injection. In a further embodiment, the compositions are administered by intramuscular injection. The compositions may be administered in one injection or in multiple injections.

Having been generally described herein, the follow examples are provided to further illustrate this invention.

### Materials and Methods

### Cells and Reagents

Prostate cancer LNCaP cells were purchased from American Type Culture Collection (ATCC, Rockville, Md.) and maintained in RPMI 1640 medium containing 10% FBS (Gibco, Grand Island, N.Y.) at 5% CO₂ and 37° C. The PSA promoter luciferase reporter plasmid (pGL3-PSA-luc), control vector and internal control Renilla luciferase vector (pRL-CMV-luc) were provided by Dr. Gerry Coetzee (University of Southern California, Los Angeles, Calif.). Human LH was purchased from Fitzgerald Industries Intl. (Concord, Mass.). siRNA targeting human LHR (LHR-siRNA) and a scrambled siRNA sequence (AllStars negative control siRNA) were designed and synthesized by Qiagen (Carlsbad, Calif.) with the target sequence of CTGAGTTAGAATTACTCTGAA (SEQ ID NO.: 1). All PCR primers were purchased from Invitrogen (Carlsbad, Calif.).

LHR Silencing by siRNA Transfection

LNCaP cells were plated at a density of 10⁵ cells/6-well plates in RPMI 1640 with 5% FBS without antibiotics two days before the transfection. The cells were transfected in serum- and antibiotic-free RPMI 1640 with the transfection reagent HiPerFect according to the manufacturer's instructions (Qiagen). After 24 hours of transfection, transfection medium in each well was replaced by normal RPMI 1640 with 5% FBS. The cells were maintained in culture for 4 days and LHR knockdown expression in the transfected cells was analyzed by Western blot with a primary anti-LHR rabbit polyclonal antibody (1:200; Santa Cruz Biotech, Santa Cruz, Calif.). A scrambled siRNA sequence was used as a negative control. Transfection efficiency (>95%) was calculated by transfection with FITC conjugated-control siRNA (Santa Cruz Biotech).

### Transient Transfection and Luciferase Assays

LNCaP cells were plated at a density of 2×10⁴ cells/48-well plates in RPMI 1640 with 5% FBS without antibiotics two days before the transfection. The cells were co-transfected in triplicate for 24 hours with the PSA promoter luciferase reporter plasmid (pGL3-PSA-luc) and an internal control Renilla luciferase vector (pRL-CMV-luc) using the Lipofectamine 2000 transfection reagent (Invitrogen). The transfected cells were treated with human LH in RPMI 1640 with 1% FBS for two days. Luciferase activities were measured by using the dual-luciferase reporter gene assay system (Promega Co., Madison, Wis.) following the manufacturer's instruction. Final results were normalized for transfection efficiencies using the Renilla luciferase assay value.

### Real-Time PCR Analysis

The quantitative measurement of target mRNA was performed using a real-time PCR system (Applied Biosystems 7900; Foster, Calif.) according to the manufacturer's specifications. PCR amplifications were performed with the SYBR Green PCR core reagent (Applied Biosystems) in a volume of 10 μA, with 1 μl of the reverse transcription products. LH and steroidogenic enzyme RNA quantification were assayed with the following primers: AKR1C1, 5′-attcccatcgaccagagttg-3′ (forward), 5′-tttgggatcacttcctcacc-3′ (reverse); AKR1C2,5′-gatcccatcgagaagaacca-3′ (forward), 5′-acacctgcacgttctgtctg-3′ (reverse); AKR1C3,5′-atttggcacctatgcacctc-3′ (forward), 5′-cacactgccatctgcaatct-3′ (reverse); CYB5,5′-gaagagcctgctttggacac-3′ (forward), 5′-aaatttgagcgcagaaagga-3′ (reverse); CYP11A1,5′-gcaacgtggagtcggtttat-3′ (forward), 5′-aggggcaaaaagttcttggt-3′ (reverse); CYP17A1,5′-gagttcgagaccagcctgac-3′ (forward), 5′-gcttctcgggttcaagtgag-3′ (reverse); FASN, 5′-cccacctacgtactggccta-3′ (forward), 5′-cttggccttgggtgtgtact-3′ (reverse); HSD3B2,5′-tggactcctctgtccagctt-3′ (forward), 5′-ctagcgtgacccagaagagg-3′ (reverse); HSD17B2,5′-ggcaactcaagctcaaggac-3′ (forward), 5′-actcagcgtggcttcttcat-3′ (reverse); HSD17B3,5′-ttttgctgctgttgttcctg-3′ (forward), 5′-gatcgcactactgcactcca-3′ (reverse); RDH5,5′-cagcaatgcctttgtcttca-3′ (forward), 5′-taccagccacaccagcatta-3′ (reverse); SRD5A1,5′-tcgcatgaaaacttgcgtag-3′ (forward), 5′-ttgaagttccacagccactg-3′ (reverse); SRD5A2,5′-gccctctcctcatagtgctg-3′ (forward), 5′-ccaggttcatgcctttttgt-3′ (reverse); StAR, 5′-ggctactcagctcgacctc-3′ (forward), 5′-catcccactgtcaccagatg-3′ (reverse); AR, 5′-taccagctcaccaagctcct-3′ (forward), 5′-gcttcactgggtgtggaaat3′ (reverse); PSA, 5′-ccaagttcatgctgtgtgct-3′ (forward), 5′-agggagttgataggggtgct-3′ (reverse); and LHR, 5′-tcaattcttgtccaatcca-3′ (forward), 5′-ccatttttgcagttggaggt-3′ (reverse). Each gene under each condition was amplified in triplicate. Analysis was performed with Applied Biosystems' software and the relative expression was standardized using expression of 18S as a reference: 5′-ggagagggagcctgagaaac-3′ (forward), 5′-tcgggagtgggtaatttgc-3′ (reverse). Results were plotted as the mean±SD from three experiments.

### Western Blot Analysis

Equal amounts of protein (10 μg) from cell lysates were heated at 95° C. for 5 min in the sample-loading buffer, then subjected to SDS-PAGE and transferred to nitrocellulose membranes. The blots were probed overnight at 4° C. with the appropriate commercially available primary antibodies, including anti-LHR rabbit polyclonal antibody (1:500; Santa Cruz Biotech), anti-AR mouse monoclonal antibody (1:1000; DAKO, Carpinteria, Calif.), anti-p-AKT mouse monoclonal antibody (1:500, Santa Cruz Biotech), anti-AKT rabbit polyclonal antibody (1:1000, Santa Cruz Biotech), anti-p-PKAc rabbit polyclonal antibody (1:1000, Cell Signaling, Danvers, Mass.), anti-PKA rabbit polyclonal antibody (1:500, Santa Cruz Biotech), anti-p-ERK1/2 mouse monoclonal antibody (1:500, Cell Signaling), anti-ERK1/2 rabbit polyclonal antibody (1:1000, Cell Signaling), anti-HER-2 mouse monoclonal antibody (1:1000, DAKO), anti-PSA rabbit polyclonal antibody (1:1000, DAKO), anti-PCNA mouse monoclonal antibody (1:1000, Santa Cruz Biotech), Anti-PI3K rabbit polyclonal antibody (1:500, Cell Signaling), anti-CYP-17A1 rabbit polyclonal (1:400; Abcam, Cambridge, Mass.), anti-CYP-11A1 rabbit polyclonal antibody (1:400; Abcam), and anti-StAR mouse monoclonal antibody (1:500; Abcam). After incubation with the corresponding horseradish peroxidase-conjugated secondary antibodies, the blots were further probed with corresponding HRP-conjugated antibodies and visualized by enhanced chemiluminescence (Pierce, Rockford, Ill.). Rabbit anti-β-actin antibody (1:2,000; Sigma Chemical Co., St. Louis, Mo.) served as a loading control. Quantification of the bands was performed with Quantity One software (Bio-Rad Lab, Hercules, Calif.) with normalization to β-actin. Protein expression analysis was replicated in at least three separate experiments to confirm results.

### Immunohistochemistry

Deparaffinization of PCA tissue was performed with xylene and tissue was rehydrated in graded ethanol solutions and rinsed in water. The slides were buffered with dilute hydrogen peroxide and blocked with 20% fetal bovine serum, then incubated overnight at 4° C. with anti-LHR rabbit polyclonal antibody (Santa Cruz Biotech). The tissue was then incubated for 1 hour at room temperature with the secondary rabbit anti-mouse antibody (1:1000; Dako). The slides were developed with diaminobenzidine tetrahydrochloride solution (Dako), lightly counterstained with hematoxylin and cover slipped. Samples from 30 patients were subject to analysis.

### Steroid Analysis by Radioimmunoassay (RIA)

Concentrations of testosterone and dihydrotestosterone (DHT) were determined by radioimmunoassay as previously described. Purification involved extraction of steroids using ethyl acetate:hexane (3:2) and subsequent separation of progresterone from testosterone by Celite column partition chromatography. The progesterone and testosterone fractions were then subjected to RIA using highly specific antiserum against each steroid together with the appropriate iodinated radioligand. Antibody bound and unbound fractions were separated by use of a second antibody. Steroid levels were measured using cell fractionation. Data were normalized per 10⁶ cells and expressed as the mean±SD from three 100 ml cell culture dishes per group.

### Cell Viability (MTS) Assay

Cells were treated with LH at varying concentrations and incubated with 20 μl of Celltiter 96® Aqᵤₑₒᵤₛ One Solution Cell Proliferation assay (Promega) according to the manufacturer's instructions. The optical density was determined using a microplate reader (SpectraMax Plus) at 490 nm. The cell viability following LH treatment was expressed versus negative control and plotted as the mean±SD.

### Apoptotic Analysis by TUNEL Assay and Caspase-3 Detection

TUNEL assay (terminal deoxynucleotidyl transferase-mediated dUTP-biotin nick end labeling) was performed to quantify the relative numbers of apoptotic cells. LNCaP cells were plated in slide chambers (10⁵/chamber). After treatment, cells were examined for apoptosis by DeadEnd™ Colorimetric TUNEL System according to the manufacturer's protocol (Promega, Madison, Wis.). The labeled cells were examined using a light microscope and cells were counted in 10 randomly chosen fields (200×). Results were expressed as the percentage of TUNEL-positive cells/total number of cells counted. Caspase-3 activity was determined by colorimetric assay based on the hydrolysis of acetyl-Asp-Glu-Val-Asp p-nitroanilide (Ac-DEVD-pNA) by caspase 3 (Sigma-Aldrich., St Louis, Mo.). LNCaP Cells (5×10⁵ perwell) were seeded in a E-well plate. After 5 days of LHR-siRNA transfection, casapse-3 colorimetric activity (OD) was quantified using a plate reader at 405 nm as per manufacturer's instruction.

### Statistics

Results were reported as means±SD of at least three experiments. The student t-test was used for statistical analyses and the differences between two means with a p-value<0.05 were considered significant.

### Results

### Functional LHR is Expressed in LNCaP Cells and Suppressed by LHR-siRNA

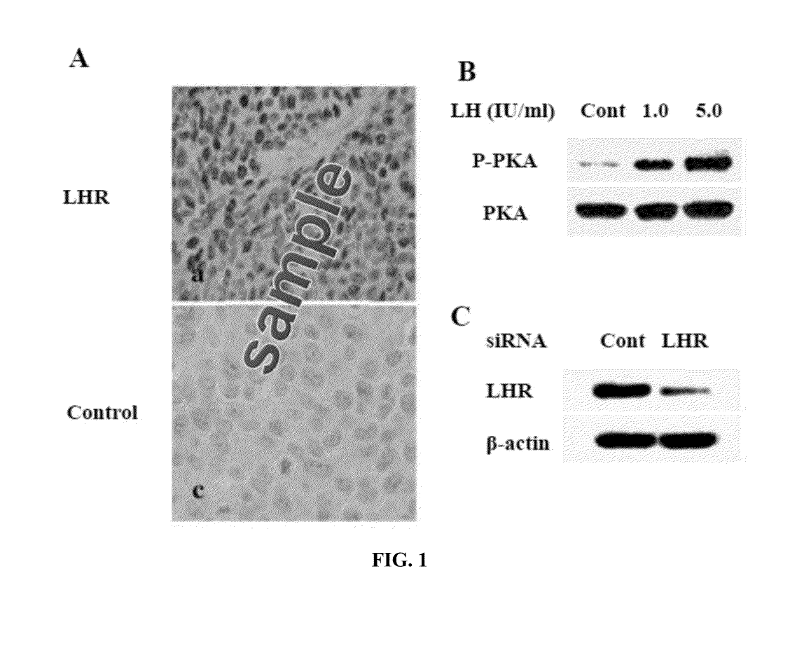

Applicant first independently confirmed LHR expression in clinical specimens. In a cohort of 15 patients with prostate cancer, formalin-fixed paraffin embedded prostatectomy samples were subject to immunohistochemistry and demonstrated strong LHR expression (HG. 1A). Western blot analysis also showed strong LHR expression in LNCaP cell lysate. Signaling via LHR is mediated by phosphorylation of PKA. To determine whether these receptors were functional, these LNCaP cells were treated with LH and changes in PKA phosphorylation were measured by Western blot analysis. There was minimal phosphorylation of PKA prior to treatment and phosphorylation increased after LH exposure in a dose-dependent manner (FIG. 1B). LHR-siRNA was then used to suppress LHR expression in LNCaP cells. Transfection efficiency was above 95% as accessed by transfection with FITC conjugated-control siRNA. After 4 days of transfection with LHR-siRNA, expression was significantly downregulated as compared to untreated controls (FIG. 1C).

### Silencing LHR Suppressed Cell Proliferation and Induces Apoptosis

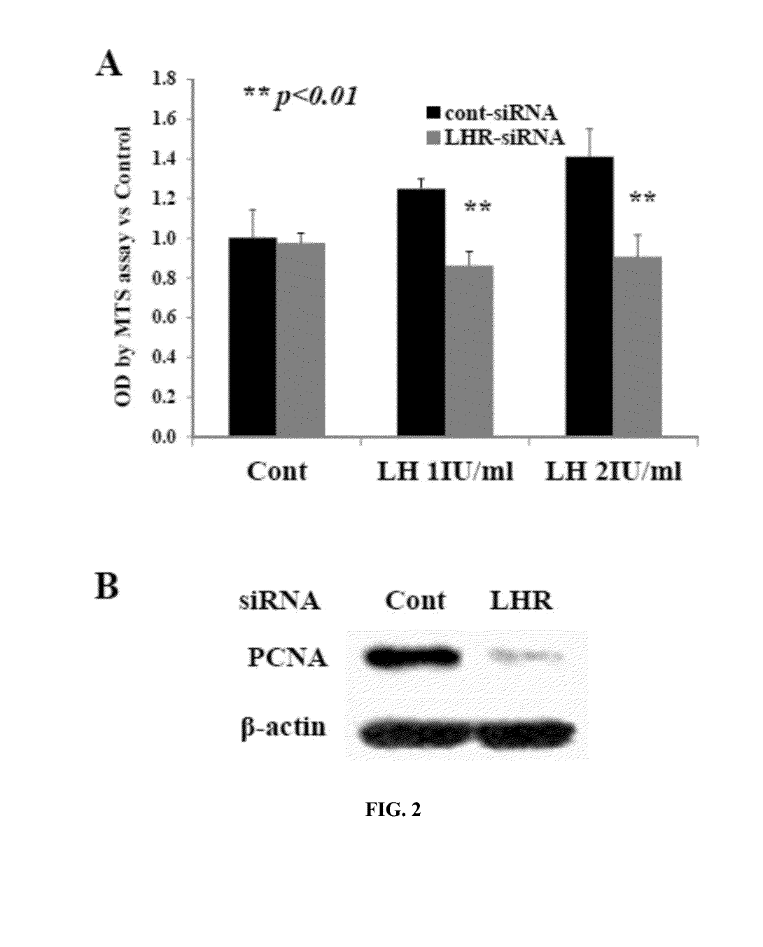

LNCaP cells were transfected with LHR-siRNA or scrambled siRNA for five days, then exposed to LH. Cell proliferation in response to LH was significantly suppressed after LHR silencing, as assessed by MTS assay (FIG. 2A) and by decreased expression of the cell proliferation marker, PCNA (FIG. 2B). Transfection with LHR-siRNA induced an apoptotic phenotype (FIG. 2C). Apoptosis was then accessed by TUNEL staining. The TUNEL labeled cells were counted in 10 randomly chosen fields (200×) and results were expressed as the percentage of TUNEL-positive cells/total number of cells counted. LHR-siRNA increased apoptosis as measured by TUNEL staining (p<0.05, FIG. 2D). Apoptosis was further assessed by a colorimetric caspase 3 activity assay based on the hydrolysis of acetyl-Asp-Glu-Val-Asp p-nitroanilide (Ac-DEVD pNA) by caspase 3. LHR silencing with LHR-siRNA was associated with a significant increase in caspase 3 activity (FIG. 2E) as compared to control (p<0.01).

### Silencing LHR Suppressed Androgen Production

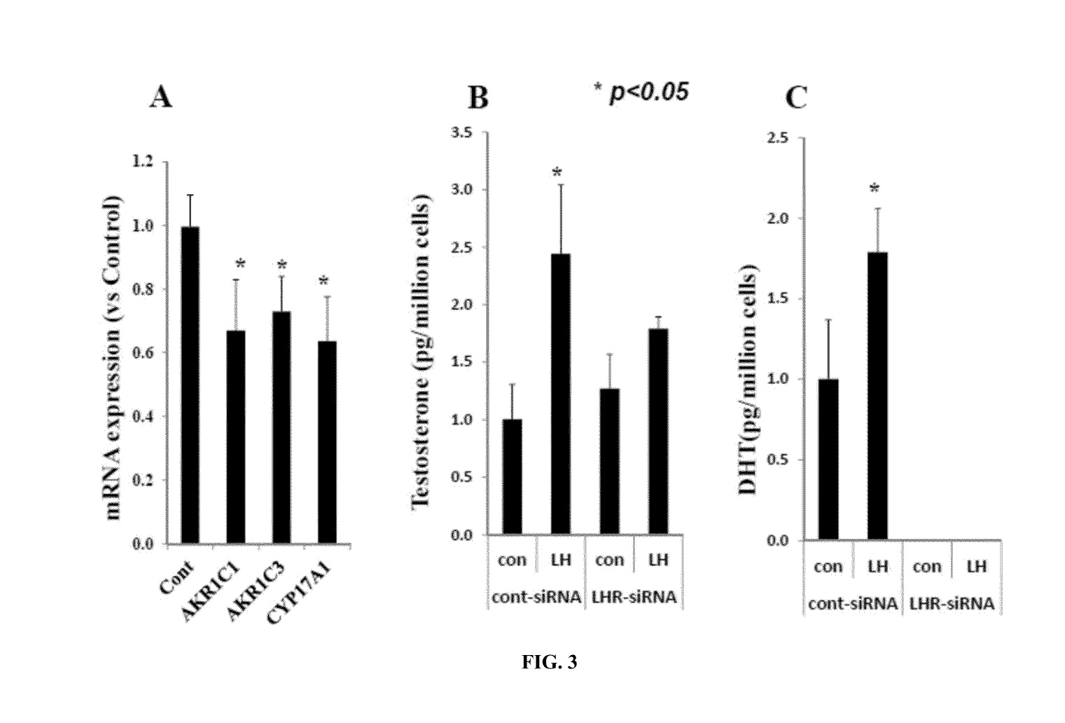

It was previously shown that LH induces androgen synthesis machinery and testosterone synthesis in LNCaP cells. Applicant sought to explore the impact of LHR-siRNA on androgen synthesis. Using scrambled siRNA as a control, silencing LHR with LHR-siRNA suppressed mRNA expression of the steroidogenic enzymes AKR1C1, AKR1C3 and CYP17A1 as measured by real-time PCR (FIG. 3A). Using a radioimmunoassay (RIA), we confirmed that LH induced synthesis of testosterone (FIG. 3B, left) and dihydrotestosterone (FIG. 3C, left). Transfection with LHR-siRNA suppressed testosterone synthesis as compared to scrambled siRNA (FIG. 3B, right) and completely prevented dihydrotestosterone synthesis (FIG. 3C, right).

### LHR Silencing Suppressed Expression of AR and PSA

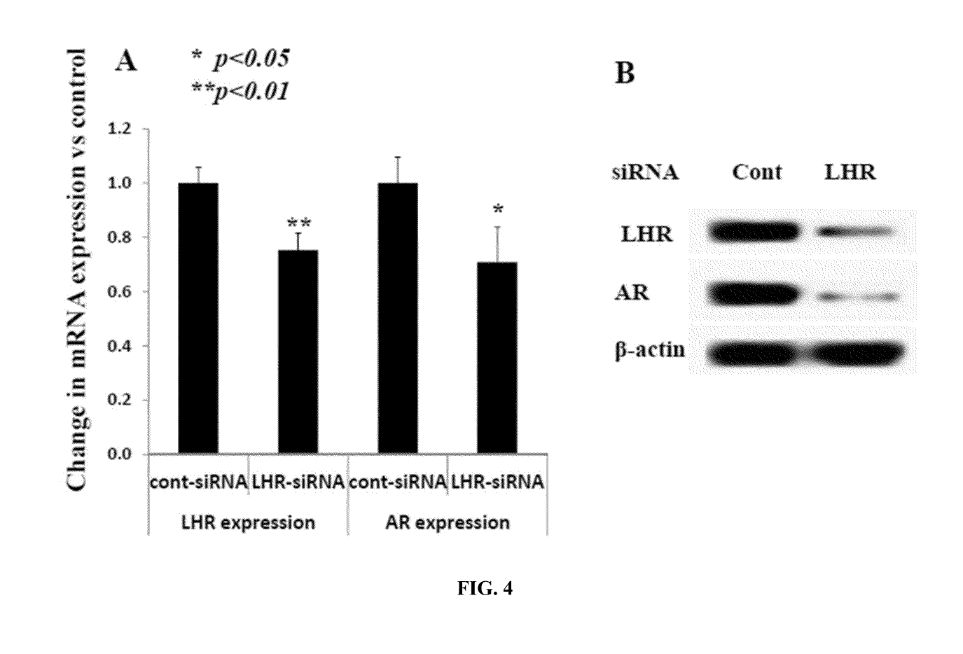

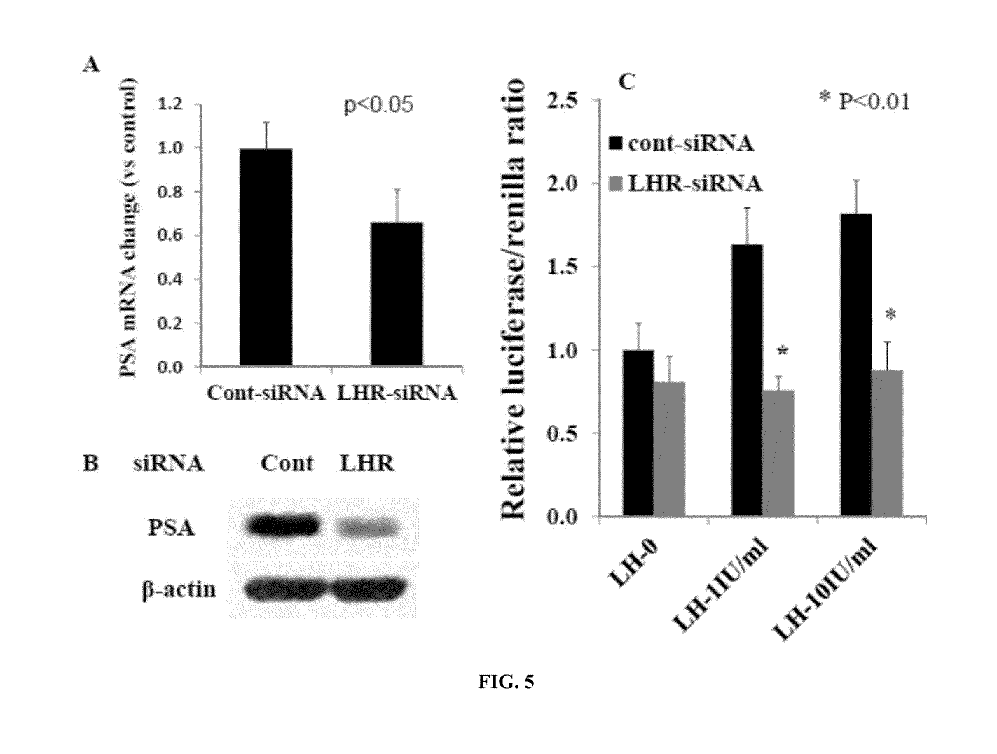

In light of the known association between expression of AR and LH in human granulose-luteal cells, we explored the effect of LHR silencing on AR expression. Transfection with LHR-siRNA significantly suppressed expression of AR mRNA (p<0.05, FIG. 4A) and protein (FIG. 4B). Ligand-activated AR is known to engage the PSA promoter and initiate expression of PSA. LHR-siRNA effectively suppressed expression of PSA mRNA (FIG. 5A) and protein (FIG. 5B). Using a luciferase/renilla assay, LHR-siRNA was also shown to suppress LH-induced PSA promoter activity (FIG. 5C).

### LHR Silencing Suppressed Activity of PKA, PI3K/AKT, ERK and Expression HER-2

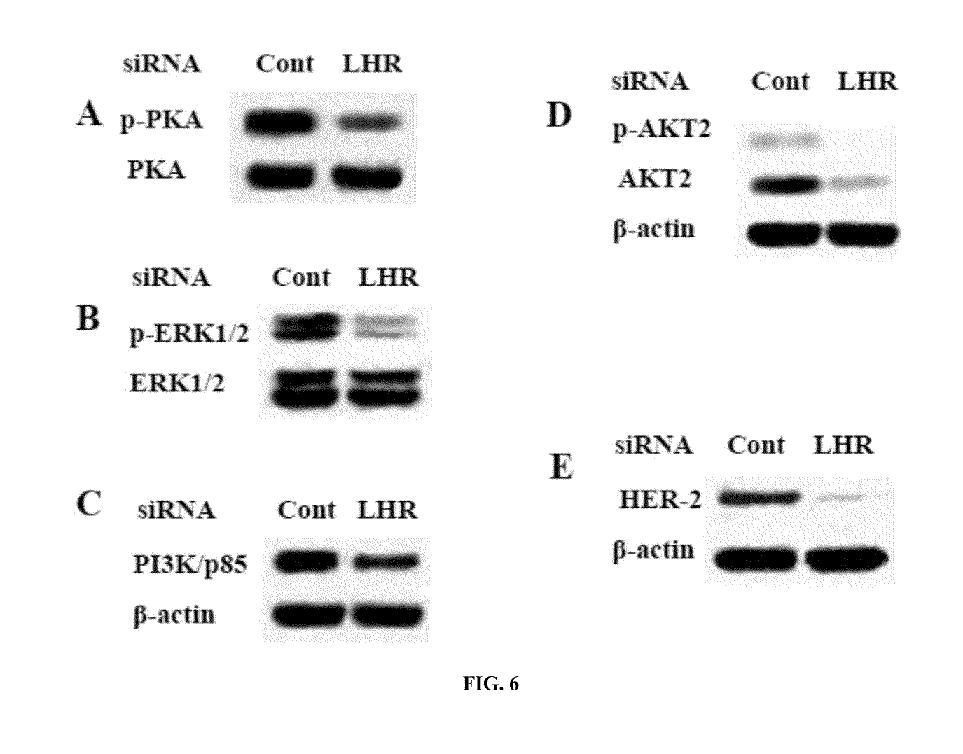

Applicant then explored the effect of LHR silencing on several key survival pathways in prostate cancer that have been implicated in castration-resistance. The PKA signal transduction pathway has been shown to activate the AR signaling pathway. Transfection of LNCaP cells with LHR-siRNA inhibits PKA phosphorylation (FIG. 6A). Similarly, LHR-siRNA also reduced phosphorylation of ERK1/2 (FIG. 6B) and the PI3K p85 subunit (FIG. 6C). LHR-siRNA also reduced expression and phosphorylation of AKT2 (FIG. 6D). Interestingly, HER2 expression was also suppressed by LHR-siRNA as compared to scrambled siRNA (FIG. 6E).

### Discussion

In this invention, Applicant implicate LHR in androgen synthesis and signaling pathways critical for prostate cancer progression. It has become increasingly apparent that androgens and androgen related gene networks remain vitally important for prostate cancer, even in the clinically castrate-resistant state. Despite low levels of circulating androgens resulting from standard androgen ablation therapy, prostate cancer cells may retain a permissive androgenic environment. Adrenal-derived androgens may be present in quantities that remain below the threshold of blood-based detection assays but are still fully capable of activating essential signaling cascades. In addition, recent data has identified the prostate cancer cell itself as a source of these androgens in a self-sufficient autocrine and paracrine pathway. Prostate cancer cells express the enzymatic machinery necessary for androgen synthesis and the expression of these enzymes is upregulated in the castrate-resistant state. In light of these findings, androgen metabolism and signaling pathways have emerged as a rational therapeutic target in CRPC, leading to the development of several potent agents that act within this network. This includes tertiary hormone manipulation with enzalutamide, an AR antagonist, which is active in patients with CRPC, and agents targeting androgen synthesis via the CYP17 pathway, including abiraterone, an inhibitor of CYP17 hydroxylase, and orteronel (TAK-700), another potent inhibitor of the CYP17 pathway that it is highly selective for CYP17,20 lyase.

While the androgen pathway is still critically active in CRPC, its regulation remains undefined. Because LH mediated signaling initiates testicular androgen synthesis, we have explored the role of this signaling pathway in tumoral androgen synthesis. We recently reported the effect of LH treatment on various prostate cancer cell lines. Exposure to LH increases cell viability and significantly increases the expression of multiple steroidogenic enzymes within prostate cancer cells, ultimately resulting in testosterone synthesis. Here, we confirm that this effect is mediated by LHR. Silencing LHR with siRNA prevents LH-induced cell proliferation and survival and prevents the upregulation of steroidogenic machinery within prostate cancer cells. LHR-siRNA suppresses LH-mediated testosterone synthesis and completely prevents production of DHT. In addition, when LHR is silenced, there is a significant downregulation of AR mRNA and protein expression with a subsequent suppression of PSA expression. To our knowledge, this link between LHR signaling and AR expression has never been reported.

There are several important signaling pathways involved in prostate cancer cell survival in the castrate resistant state, including PKA and MAPK mediated signaling. After PKA is activated, its catalytic unit phosphorylates its target proteins and kinases, such as ERK1/2 and leads to the upregulation of steroidogenesis. In the granulosa cells, LH-LHR signaling activates PI3K through upregulating RAS, and further activates AKT, a downstream target of PI3K. Activated AKT then promotes cell survival through two distinct pathways inhibition of apoptosis by phosphorylating the Bad component of the Bad/Bcl-X_{L} complex and activation of IKK-a and NF-kb, promoting cell survival. Silencing LHR leads to suppression of these signaling pathways, with decreased phosphorylation of PKA, ERK1/2 and PI3K/p85 and decreased expression and phosphorylation of AKT2. Interestingly, LHR-siRNA also downregulated expression of HER2. The LH-LHR signaling pathway may play a role in promoting a castration-resistant phenotype by triggering androgen synthesis and activating several critical survival pathways.

These data suggest that the LH-LHR signaling cascade may be an upstream and viable therapeutic target in CRPC that has remained largely unexplored. Standard androgen ablation with an LHRH agonist suppresses LH release however these levels are not likely to reflect LH concentrations at the tumoral level. As with testosterone, small concentrations of LH may be sufficient to initiate signaling cascades. Adequate concentrations of LH may be produced by prostate cancer cells or stromal cells within the microenvironment and this can be adequate to fuel progression. Ectopic LH and LHR expression have been observed in recent years, implying an autocrine or paracrine regulation. Implicating LH and LHR in the regulation of this pathway provides rational therapeutic targets for the treatment of advanced prostate cancer.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Simoni M, Gromoll J, Nieschlag E. The follicle-stimulating hormone receptor: Biochemistry, molecular biology, physiology, and pathophysiology. Endocr Rev 1997; 18:739-773.
2. De Luca A, Maiello M R, D'Alessio A, Pergameno M, Normanno N. Expert Opin Ther Targets. 2012 April; 16 Suppl 2:S17-27. Epub 2012 Mar. 23. The RAS/RAF/MEK/ERK and the PI3K/AKT signalling pathways: role in cancer pathogenesis and implications for therapeutic approaches.
3. Sheppard K, Kinross K M, Solomon B, Pearson R B, Phillips W A. Crit. Rev Oncog. 2012; 17(1):69-95. Targeting PI3 kinase/AKT/mTOR signaling in cancer.
4. Chiaradonna F, Balestrieri C, Gaglio D, Vanoni M. Front Biosci. 2008 May 1; 13:5257-78. RAS and PKA pathways in cancer: new insight from transcriptional analysis.
5. Attard G, Richards J, de Bono J S. Clin Cancer Res. 2011 Apr. 1; 17(7):1649-57. Epub 2011 Mar. 3. New strategies in metastatic prostate cancer: targeting the androgen receptor signaling pathway.
6. Ménard S, Casalini P, Campiglio M, Pupa S M, Tagliabue E. Cell Mol Life Sci. 2004 December; 61(23):2965-78. Role of HER2/neu in tumor progression and therapy.

What is claimed is:

1. A method for one or more of:
a. downregulating androgen receptor (AR) mRNA expression and/or AR protein expression;
b. suppressing prostate (PSA) expression and/or PSA promoter activity;
c. suppressing the activity of PKA;
d. suppressing the activity of PI3K/AKT;
e. suppressing the activity of ERK; or
f. suppressing the expression of HER-2,
in a cell expressing luteinizing hormone receptor (LHR), comprising contacting the cell with an effective amount of an agent that inhibits the expression of LHR.

2. The method of claim 1, wherein the contacting is in vitro or in vivo.

3. The method of claim 1 or 2, wherein the agent that inhibits the expression of LHR is of the group of: a small molecule inhibitor, an inhibitory RNA molecule or an anti-LHR antibody, fragment or a derivative thereof.

4. A method for treating a cancer or tumor cell expressing luteinizing hormone receptor (LHR), comprising contacting the cell with an effective amount of an agent that inhibits the expression of LHR.

5. The method of claim 4, wherein the cancer or tumor is of the group of prostate cancer, castration-resistant prostate cancer (CRPC), ovarian cancer or breast cancer.

6. The method of claim 4 or 5, wherein the agent that inhibits the expression of LHR is of the group of: a small molecule inhibitor, an inhibitory RNA molecule or an anti-LHR antibody, a fragment or a derivative thereof.

7. A method to screen for potential therapeutic agents that possess one or more of:
a. downregulating androgen receptor (AR) mRNA expression and/or AR protein expression;
b. suppressing prostate (PSA) expression and/or PSA promoter activity;
c. suppressing the activity of PKA;
d. suppressing the activity of PI3K/AKT;
e. suppressing the activity of ERK; or
f. suppressing the expression of HER-2,
comprising contacting a first cell expressing LHR with a potential therapeutic agent and noting the expression or activity of the expression or activity of one or more of a. to f., with that of a second cell expressing LHR having been contacted with an agent that inhibits the expression of LHR, wherein if the expression or activity of the first cell is the same or better than that of the second cell, the agent is a potential therapeutic agent.

8. A kit useful for one or more of:
a. androgen receptor (AR) mRNA expression and/or AR protein expression
b. prostate (PSA) expression and/or PSA promoter activity;
c. the activity of PKA;
d. the activity of PI3K/AKT;
e. the activity of ERK; or
f. the expression of HER-2,
comprising an effective amount of an agent that inhibits the expression of LHR and optionally instructions for use.

9. The method of claim 8, wherein the agent that inhibits the expression of LHR is of the group of a small molecule inhibitor, an inhibitory RNA molecule, an anti-LHR antibody or a fragment or derivative thereof.

10. A method for treating a cancer expressing luteinizing hormone receptor (LHR) in a subject in need thereof, comprising administering to the subject an effective amount of an agent that inhibits the expression of LHR, thereby treating the cancer.

11. The method of claim 10, wherein the cancer is of the group of prostate cancer, castration-resistant prostate cancer (CRPC), ovarian cancer or breast cancer.

12. The method of claim 10 or 11, wherein the agent that inhibits the expression of LHR is of the group of: a small molecule inhibitor, an inhibitory RNA molecule or an anti-LHR antibody, a fragment or a derivative thereof.

13. The method of claim 10, wherein the subject is a human patient.

14. The method of claim 12, wherein the agent is an anti-LHR antibody fragment.

15. The method of claim 14, wherein the anti-LHR antibody fragment further comprises one or more of the group of: a radiolabel, a cytokine or a co-stimulatory molecules.

16. The method of claim 15, further comprising a linker position between the anti-LHR antibody fragment and the one or more of the group of: a radiolabel, a cytokine or a co-stimulatory molecules.

17. The method of claim 16, wherein the linker is a labile or non-labile linker.
